(19) United States
(12) Patent Application Publication
(10) Pub. No.: US 2021/0025012 A1
(43) Pub. Date: Jan. 28, 2021

(54) METHOD FOR DETECTING NUCLEIC ACID

(71) Applicant: Shimadzu Corporation, Kyoto (JP)

(72) Inventors: Takaoka Naoko, Kyoto (JP); Shikata Masamitsu, Kyoto (JP); Ninomiya Kenji, Kyoto (JP); Kobayashi Shinichiro, Kyoto (JP)

(21) Appl. No.: 16/929,032

(22) Filed: Jul. 14, 2020

(30) Foreign Application Priority Data
Jul. 22, 2019 (JP) 2019-134717

(51) Int. Cl. C12Q 1/6888; C12Q 1/686; C12Q 1/6806

(52) U.S. Cl. CPC C12Q 1/6888; C12Q 1/686; C12Q 1/6806; C12Q 2561/113; C12Q 2521/101; C12Q 2523/32; C12Q 2563/107; C12Q 2521/107

(57) ABSTRACT

The present invention relates to a method for detecting an RNA virus in a specimen by reverse transcription-polymerase chain reaction (RT-PCR) and a kit for carrying out the method. More specifically, the present invention relates to a test method, characterized in that the purified water, saline or a buffer for mixing with a specimen to obtain a centrifugation supernatant as an analysis sample previously contain at least one element selected from internal control DNA, forward and reverse primers that specifically hybridize to the DNA, while RT-PCR reaction solution does not contain the above-mentioned element(s) contained in the purified water, saline, or buffer, and a kit for carrying out the method.

### TECHNICAL FIELD

The present invention relates to a method for detecting an RNA virus in a specimen by reverse transcription-polymerase chain reaction (RT-PCR) and a kit for carrying out the method. More specifically, the present invention relates to a test method, characterized in that the purified water, saline or a buffer for mixing with a specimen to obtain a centrifugation supernatant as an analysis sample previously contain at least one element selected from internal control DNA, forward and reverse primers that specifically hybridize to the DNA, while RT-PCR reaction solution does not contain the above-mentioned element(s) contained in the purified water, saline, or buffer, and a kit for carrying out the method.

### BACKGROUND TECHNOLOGY

In order to prevent infection and spread of infection by bacteria or viruses, it is important to identify persons infected with the bacteria or virus or contaminants by the bacteria or virus. The contaminants include feces or vomitus of the infected persons and articles directly or indirectly contaminated with these, as well as foods contaminated with bacteria or viruses.

Although there are various methods for detecting bacteria or viruses, the nucleic acid detection method by PCR has been widely used as a method for rapid measurement. For example, as a mean for rapidly and highly sensitively measuring norovirus, which is a RNA virus, there is a method of amplifying Norovirus RNA by RT-PCR and measuring the amount of the amplified product (Patent Documents 1 and 2, Non-Patent Document 1). In Japan, detection of norovirus by the RT-PCR method and quantitative detection of norovirus by the real-time PCR method are widely conducted in accordance with the notification by the Food Safety Division, Drug and Food Department, Ministry of Health, Labor and Welfare (Non-patent documents 2 and 3). The norovirus detection kit is commercially available.

If the subject to be tested for bacterial or viral contamination is feces or other feces, usually, a fecal emulsion obtained by suspending feces in purified water or saline is subjected to high-speed centrifugation, and the obtained supernatant is used as a specimen to analyze genes derived from microorganisms by PCR or RT-PCR. Generally, commercially available nucleic acid test kits contain internal control DNA, when added to the PCR reaction system, it is used as an index for judging whether or not the detection of the nucleic acid is appropriately performed, that is, whether or not the PCR reaction has proceeded appropriately. As a result, even when the amplification curve or the melting curve peak of the amplification product for the nucleic acid derived from the microorganism in the specimen is not detected, if an amplification curve of the internal control DNA or a melting curve peak of the amplification product is detected, it is judged to be a true negative for microbial contamination. Further, when the PCR reaction is inhibited by the component derived from the sample, nucleic acid derived from the microorganism in the specimen is not detected, when the internal control DNA coexists, the internal control DNA is also not detected, so that it is possible to avoid a situation where the microbial contamination of the specimen is erroneously determined to be negative (false negative).

### PRIOR ART DOCUMENT

### Patent Document

1. WO2002/029119

2. WO2002/029120

### Non-Patent Document

1. Kageyama T, et al. Broadly reactive and highly sensitive assay for Norwalk-like viruses based on real-time quantitative reverse transcription-PCR. J Clin Microbiol. 2003 April; 41(4):1548-57.
2. Food Safety Division, Drug and Food Department, Ministry of Health, Labor and Welfare Food, Food Safety Audit No. 1105001 (Nov. 5, 2003) Attachment “Detection Method of Norovirus”, final revision: Food Safety Audit No. 0514004 (May 14, 2007)
3. Food Safety Division, Drug and Food Department, Ministry of Health, Labor and Welfare Food, Food Safety Audit No. 1105001 (Nov. 5, 2003) Attachment “Detection Method of Norovirus”, final revision: Food Safety Audit 1022 No. 1 (Oct. 22, 2013)

### SUMMARY OF THE INVENTION

### Problems to be Solved by the Invention

Usually, in a nucleic acid test kit, all of the elements that allow the PCR reaction to proceed, such as template DNA as an internal control and primers, are contained in the PCR reaction solution or the RT-PCR reaction solution. Therefore, even if the specimen to be tested for microbial contamination is not added to the PCR reaction solution or RT-PCR reaction solution due to human error in analysis work, etc. since the amplification curve peak of the internal control DNA or the melting curve peak of the amplification product is detected, the test result is negative even if the gene of the microorganism is present in the specimen to be analyzed, leading to a false determination (false negative).

An object of the present invention is to provide a method for preventing a false negative in which a specimen is not added to a PCR reaction solution or an RT-PCR reaction solution due to human error, and as a result, microbial contamination of the specimen is determined to be negative, and a test kit for carrying out the method.

### Means for Solving the Problem

The object of the present invention is achieved by the following inventions.
[1] A method for detecting RNA virus in a specimen, comprising
   (1) a step of suspending the specimen in purified water, saline or buffer which contain at least one element selected from the group consisting of internal control DNA, forward and reverse primers that specifically hybridize to the DNA;
   (2) a step of extracting the centrifugal supernatant of the suspension obtained in the step (1);
   (3) a step of mixing the centrifuge supernatant extracted in the step (2) with specimen processing liquid;
   (4) a step of mixing the mixed solution obtained in the step (3) with 1-step RT-PCR reaction solution, which contains reverse transcriptase and DNA polymerase, but does not contain the element(s) selected in the step (1), and performing RT-PCR; and
   (5) a step of detecting the RT-PCR product.
[2] The method according to [1], wherein the specimen is derived from samples selected from the group consisting of biological samples, biologically derived samples, environmental samples, and environmentally derived samples.
[3] The method according to [1], wherein the specimen is derived from samples selected from the group consisting of excretion samples, samples derived from excretion, vomitus and samples derived from vomitus.
[4] The method according to [1], wherein the RNA virus is norovirus.
[5] The method according to [4], wherein the genotype of the norovirus is genogroup I (GI) or genogroup II (GII).
[6] The method according to [1], wherein the specimen processing liquid in the step (3) contains at least one type of surfactant.
[7] The method according to [6], wherein the surfactant is an anionic surfactant.
[8] The method according to [7], wherein the anionic surfactant is selected from the group consisting of alkyl sulphates, alkyl ether sulphates, docusates, sulphonate fluorosurfactants, alkylbenzene sulphonates, alkylaryl ether phosphates, alkyl ether phosphates, alkylcarboxylates, sodium lauroyl sarcosine, carboxylate fluorosurfactants, sodium cholate and sodium deoxycholate.
[9] The method according to [7], wherein the anionic surfactant is an alkyl sulphate.
[10] The method according to [9], wherein the alkyl sulfate is sodium dodecyl sulfate or ammonium dodecyl sulfate.
[11] The method according to any one of [6] to [10], wherein the concentration of the surfactant is 0.02 to 0.5% (w/v).
[12] The method according to [1], wherein the specimen processing liquid in the step (3) contains a hydroxide.
[13] The method according to [12], wherein the hydroxide is sodium hydroxide or potassium hydroxide.
[14] The method according to [12] or [13], wherein the concentration of the hydroxide is 10 to 100 mM.
[15] The method according to any one of [1] to [14], wherein the mixing ratio of the centrifugal supernatant and the specimen processing liquid in the step (3) is 1:3 to 6 as a volume ratio.
[16] The method according to [1], wherein the specimen processing liquid in the step (3) is the sample treatment reagent contained in the norovirus detection reagent kit (probe method) (Shimadzu Corp., product No. 241-09325 series).
[17] The method according to [1], wherein the 1-step RT-PCR reaction solution in the step (4) is a mixture of NoV Reagents A, B and C contained in the norovirus detection reagent kit (probe method) (Shimadzu Corp., product No. 241-09325 series), which does not contain the element(s) selected in the step (1).
[18] The method according to [1], wherein the reverse transcriptase is selected from the group consisting of AMV reverse transcriptase, MMLV reverse transcriptase, HIV reverse transcriptase and variants thereof
[19] The method according to [1], wherein the DNA polymerase is selected from the group consisting of Taq DNA polymerase, Tth DNA polymerase, KOD DNA polymerase, Pfu DNA polymerase and variants thereof
[20] The method according to [1], wherein detection of the RT-PCR product in the step (5) is monitored by real-time determination.
[21] The method according to [20], wherein in the real-time determination, the presence of RNA in a specimen is judged to be positive or negative by measuring the peak in the amplification curve or melting curve of the RT-PCR product using a fluorescent filter.
[22] The method according to [20] or [21], wherein in the real-time determination, whether a specimen needs to be retested is judged by measuring the peak in the amplification curve or melting curve of the PCR product against the internal control DNA.
[23] A kit for detecting RNA virus in a specimen, comprising
   (1) purified water, saline or buffer which contain at least one element selected from the group consisting of internal control DNA, forward and reverse primers that specifically hybridize to the DNA;
   (2) specimen processing liquid; and
   (3) 1-step RT-PCR reaction solution which contains reverse transcriptase and DNA polymerase, but does not contain the element(s) selected in the step (1).
[24] The kit according to [23], wherein the reverse transcriptase is selected from the group consisting of AMV reverse transcriptase, MMLV reverse transcriptase, HIV reverse transcriptase and variants thereof
[25] The kit according to [23], wherein the DNA polymerase is selected from the group consisting of Taq DNA polymerase, Tth DNA polymerase, KOD DNA polymerase, Pfu DNA polymerase and variants thereof
[26] The kit according to [23], wherein the specimen processing liquid contains at least one type of surfactant.
[27] The kit according to [26], wherein the surfactant is an anionic surfactant.
[28] The kit according to [27], wherein the anionic surfactant is selected from the group consisting of alkyl sulphates, alkyl ether sulphates, docusates, sulphonate fluorosurfactants, alkylbenzene sulphonates, alkylaryl ether phosphates, alkyl ether phosphates, alkylcarboxylates, sodium lauroyl sarcosine, carboxylate fluorosurfactants, sodium cholate and sodium deoxycholate.
[29] The kit according to [27], wherein the anionic surfactant is an alkyl sulphate.
[30] The kit according to [29], wherein the alkyl sulfate is sodium dodecyl sulfate or ammonium dodecyl sulfate.
[31] The kit according to [23], wherein the specimen processing liquid contains a hydroxide.
[32] The kit according to [31], wherein the hydroxide is sodium hydroxide or potassium hydroxide.
[33] The kit according to [23], wherein the specimen processing liquid is a sample treatment reagent contained in the norovirus detection reagent kit (probe method) (Shimadzu Corp., product No. 241-09325 series).
[34] The kit according to [23], wherein the 1-step RT-PCR reaction solution is a mixture of NoV Reagents A, B and C contained in the norovirus detection reagent kit (probe method) (Shimadzu Corp., product No. 241-09325 series), but does not contain the element(s) selected in (1).

### Effects of the Invention

According to the invention, in the step (1), the purified water, saline or buffer used to suspend a specimen comprises at least one element selected from the group consisting of internal control DNA, forward and reverse primers that specifically hybridize to the DNA. On the other hand, the RT-PCR reaction solution used in the step (4) does not contain the element(s) added to the purified water, saline, or buffer used in the step (1). Therefore, amplification of the internal control DNA occurs only when the centrifugation supernatant of the suspension of the specimen and the purified water, saline or buffer is added to the RT-PCR reaction solution. When the amplification of the internal control DNA does not occur, it means that the specimen is not provided for the RT-PCR reaction. In this way, since the specimen is not added to the RT-PCR reaction solution, an artificial error that the specimen is not correctly analyzed is detected, and a false negative determination regarding microbial contamination is prevented.

### BRIEF DESCRIPTION OF THE DRAWINGS

FIG. 1 Feces containing norovirus were suspended in distilled water containing each elements of internal control DNA, forward and reverse primers that specifically hybridize to the DNA, and the centrifugation supernatant of the suspension obtained was mixed with a specimen processing liquid. This figure is a diagram showing an amplification curve in real-time PCR performed by adding the obtained mixed solution to 1-step RT-PCR reaction solution which does not contain the element.

FIG. 2 Norovirus-free feces were suspended in distilled water containing each elements of internal control DNA, forward and reverse primers that specifically hybridize to the DNA, and the centrifugation supernatant of the suspension obtained was mixed with a specimen processing liquid. This figure is a diagram showing an amplification curve in real-time PCR performed by adding the obtained mixed solution to 1-step RT-PCR reaction solution which does not contain the element.

FIG. 3 is a diagram showing an amplification curve in real-time PCR performed by adding distilled water to 1-step RT-PCR reaction solution which does not contain internal control DNA, forward and reverse primers that specifically hybridize to the DNA.

### EMBODIMENTS FOR CARRYING OUT THE INVENTION

The present invention is a test method for detecting the presence of an RNA virus in a specimen by amplifying RNA extracted from the RNA virus in the specimen by RT-PCR. The method can prevent an artificial error in which a test is performed without adding a specimen to the measurement system causing an erroneous test result.

The RNA virus to be detected in the present invention has RNA as its genome. Their examples include, but are not limited to, coronavirus which has an envelope, a membrane composed of lipid bi-layers, human immunodeficiency virus, hepatitis C virus, Japanese encephalitis virus, dengue virus, etc., norovirus without envelope, rotavirus, rhinovirus, and the like.

Examples of the specimens in the present invention include biological samples, biologically derived samples, environmental samples and environmentally derived samples, and the like. Examples of the biological samples include animal and plant tissues including the midgut glands of shellfish and body fluids such as blood, saliva, nasal discharge, and tissue secretions. Shellfish, for example, is the most important food source of food poisoning caused by norovirus. Examples of the biologically derived samples include a sample obtained by subjecting the biological sample or a suspension thereof to treatment such as sonication. Examples of environmental samples include all samples including air, soil, dust, water and the like. Examples of the environmentally derived samples include those obtained by subjecting the environmental samples to treatment such as sonication.

As another embodiment of the present invention, examples of the specimens include excrement samples, samples derived from excrement, vomitus samples, samples derived from vomitus, body fluid samples such as saliva, and samples derived from body fluid sample. Samples derived from excrement and samples derived from vomitus include wiping samples. The wiping samples are used to confirm bacterial or viral contamination by wiping fingers, tableware, cutting boards, knives, cooking equipment, toilet equipment, housing equipment, etc. with cotton swabs, cut cotton, etc. and dissolving in a phosphate buffer or the like. The solution obtained is subjected to ultracentrifugation, and the centrifugal sediment can be used as a specimen (Keiko Soumura et al., Journal of Food Hygiene, 2017, Vol. 58, No. 4, p. 201-204).

In the step (1) of the method of the present invention, a specimen such as an excrement sample, a vomitus sample and a body fluid sample is suspended in purified water, saline or a buffer which contain at least one element selected from internal control DNA, forward and reverse primers that specifically hybridize to the DNA, at 5 to 10% (w/v) to give an emulsion or suspension. The purified water is produced from ordinary water by a system in which ion exchange, distillation, reverse osmosis, ultrafiltration or the like is used alone or in combination. The buffer is not particularly limited, and examples thereof include phosphate buffer, Tris buffer, borate buffer, Good buffer such as HEPES. The emulsion or suspension is centrifuged in the step (2) at, for example, 10000 to 12000 rpm for 2 to 20 minutes, and the obtained centrifugation supernatant is used in the step (3).

In the step (3) of the present invention, RNA can be extracted from the RNA virus contained in the specimen by using the specimen processing liquid. In one embodiment of the present invention, the specimen processing liquid used in the step (2) contains one or more surfactants. As used herein, the term “surfactant” is a general term for substances that act on the boundary surface of substances and change their properties. The surfactant has a structure having both a hydrophilic portion and a hydrophobic portion in the molecule. Surfactants are classified into anionic surfactants, cationic surfactants, amphoteric surfactants and nonionic surfactants. Examples of the anionic surfactant include, but are not limited to, alkyl sulphates, alkyl ether sulphates, docusates, sulphonate fluorosurfactants, alkylbenzene sulphonates, alkylaryl ether phosphates, alkyl ether phosphates, alkylcarboxylates, sodium lauroyl sarcosine, carboxylate fluorosurfactants, sodium cholate and sodium deoxycholate. As the alkyl sulfate, sodium dodecyl sulfate (SDS) and ammonium dodecyl sulfate are preferable, and sodium dodecyl sulfate is more preferable. Sodium dodecyl sulfate is also called sodium lauryl sulfate (Sodium Lauryl Sulfate, SLS). Examples of cationic surfactants include, but are not limited to, ethyltrimethylammonium bromide, hexadecyltrimethylammonium bromide, tetradecyltrimethylammonium bromide, and the like. Examples of amphoteric surfactants include, but are not limited to, betaine and alkylamino fatty acid salts. Examples of the nonionic surfactant include, but are not limited to, nonylphenoxy polyethoxy ethanol (NP-40), polyoxyethylene sorbitan monooleate (Tween® 80), polyoxyethylene pt-octylphenol (Triton X-100®), and the like.

When a surfactant is added at a certain concentration or higher in an aqueous solution, the surfactant monomers aggregate to form micelles. The concentration at which the surfactant becomes micelle-forming is called the critical micelle concentration. In an aqueous solution, the hydrophobic region of the protein or lipid is incorporated into the hydrophobic region inside the surfactant micelle, and the protein or lipid is solubilized. In RNA virus particles, an envelope composed of capsids and lipids that are protein shells is solubilized, denatured, or destroyed in the presence of a surfactant at a critical micelle concentration or higher. As a result, the RNA encapsulated in the capsid is likely to be exposed in the aqueous solution. The critical micelle concentration of the surfactant varies depending on the type of the surfactant, but in order to efficiently expose the viral RNA, the concentration of the surfactant in the specimen processing liquid is preferably 0.02 to 0.5% (w/v), more preferably 0.05 to 0.2% (w/v), and even more preferably 0.1% (w/v).

In the step (3) of the present invention, the mixing ratio of the centrifugal supernatant obtained in the step (2) and the specimen processing liquid is preferably 1:3 to 6, and more preferably 1:4, as a volume ratio. By mixing the centrifugation supernatant and the specimen processing liquid containing the surfactant, the concentration of the surfactant in the mixed liquid decreases, however, the above concentration of surfactant maintains the critical micelle concentration.

In one embodiment of the present invention, the specimen processing liquid contains a hydroxide. In the present specification, “hydroxide” refers to a substance in which a metal ion as a cation and a hydroxide ion (OH—) as an anion are ionically bonded. The metal is an alkali metal or an alkaline earth metal. Examples of hydroxides include lithium hydroxide, sodium hydroxide, potassium hydroxide, magnesium hydroxide, calcium hydroxide and barium hydroxide, among which sodium hydroxide and potassium hydroxide are preferred. Hydroxides are strongly basic and, when dissolved in water, produce hydroxide ions, so they are also called alkalis. Hydroxide changes the charge state of dissociative amino acids such as aspartic acid and glutamic acid in a protein molecule in an aqueous solution to denature the protein. This effect causes destruction of the capsid when the RNA virus particles are treated with alkali. As a result, the RNA encapsulated in the capsid is likely to be exposed in the aqueous solution. In order to efficiently expose viral RNA, the hydroxide concentration in the specimen processing liquid is preferably 10 to 100 mM, more preferably 40 to 60 mM, and furthermore preferably 50 mM.

In order to solubilize, denature, or destroy the envelope composed of capsids and lipids and efficiently expose viral RNA, it is preferable that the surfactant and hydroxide coexist in the specimen processing liquid.

The step (3) of the present invention for efficiently exposing viral RNA from the capsid is performed preferably at a temperature of 1 to 60° C., more preferably 1 to 50° C., and furthermore preferably 1 to 40° C., most preferably at room temperature of 1 to 30° C. After the centrifugation supernatant obtained in the step (2) and the specimen processing liquid are mixed, it is preferable to allow them to stand for 3 minutes or longer.

For RNA extraction from RNA viruses, the sample treatment reagent included in a commercially available norovirus detection reagent kit (probe method) (Shimadzu Corp., product No. 241-09325 series, 241-09325-91 or 241-09325-92) can be used. In this case, RNA can be extracted according to the instruction manual of the kit.

The specimen processing liquid for extracting RNA from RNA virus is not particularly limited as long as it does not or hardly inhibit the RT-PCR reaction even when mixed with the RT-PCR reaction solution, and can extract RNA.

In the step (3), the mixed solution of the centrifugal supernatant extracted in the step (2) and the specimen processing liquid can be heat-treated in order to increase the efficiency of RNA extraction from RNA virus. Examples of the heat treatment include heat treatment at 90° C. for 5 minutes, but the heating temperature and the heating time can be changed to improve RNA extraction efficiency.

In one embodiment of the present invention, the specimen may be RNA separated and purified from the sample. RNA can be purified by a method such as phenol extraction/water-soluble organic solvent precipitation (U.S. Pat. No. 5,527,578), precipitation from a chaotropic salt solution, or adsorption to silica. As the chaotropic salt solution, TRIzol® (Invitrogen Inc.) and ISOGEN (Nippon Gene Inc.) based on the phenol-guanidine method can be used. A commercially available spin column can be used as the method of adsorption on silica. Examples include NucleoSpin RNA® (Takara Bio Inc.) and PureLink® (ThermoFisher Inc.). There are various methods for extracting and purifying RNA in this manner, which are well known to those of skill in the art.

In one embodiment of the present invention, when RNA in a specimen is RNA separated and purified from a sample, the solution obtained in the step (1) may be immediately subjected to RT-PCR in the step (4) without carrying out the steps (2) and (3) of the present invention.

In order to detect RNA in a specimen by RT-PCR, the composition of the 1-step RT-PCR reaction solution used in the step (4) can be constructed by those skilled in the art based on well-known techniques. In one embodiment of the present invention, the reagents included in a commercially available norovirus detection reagent kit (probe method) (Shimadzu Corp., product No. 241-09325 series, 241-09325-91 or 241-09325-92) can be used. As the 1-step RT-PCR reaction solution, a mixture of NoV Reagents A, B and C contained in this kit can be used. NoV Reagent A contains magnesium ions, potassium ions and Tris. NoV Reagent B contains reverse transcription reaction primer, PCR primers for amplifying cDNA generated by reverse transcription reaction, internal control DNA, and forward and reverse primers that specifically hybridize to the DNA. However, elements selected from internal control DNA, forward and reverse primers which hybridize specifically with the DNA, and elements added to the purified water, saline or buffer used in the step (1) are removed. NoV Reagent C contains reverse transcriptase and DNA polymerase. In the 1 step RT-PCR reaction, since the reverse transcriptase and the DNA polymerase are previously mixed, the reverse transcription reaction (single-strand cDNA synthesis) and PCR can be performed in the same container.

The reverse transcriptase contained in the 1-step RT-PCR reaction solution is an enzyme that produces single-stranded complementary DNA (cDNA) using viral RNA as a template, and is not particularly limited as long as it catalyzes the reverse transcription reaction. RNA-dependent DNA polymerases derived from RNA viruses such as avian myeloblastosis virus (AMV), moloney murine leukemia virus (M-MLV) and human immunodeficiency virus (HIV) as well as variants thereof can be used.

The DNA polymerase contained in the 1-step RT-PCR reaction solution is a thermostable DNA polymerase derived from a thermophilic bacterium. Taq, Tth, KOD, Pfu and variants thereof can be used, but are not limited thereto. A hot start DNA polymerase can be used to avoid non-specific amplification by the DNA polymerase. The hot start DNA polymerase is, for example, a DNA polymerase to which an anti-DNA polymerase antibody is bound or a DNA polymerase in which an enzyme active site is thermosensitively chemically modified. It is an enzyme in which DNA polymerase is activated in PCR after the first denaturation step (90° C. or higher).

The 1-step RT-PCR reaction solution contains all components for performing reverse transcription reaction and PCR under appropriate conditions. The components include at least the reverse transcriptase, reverse transcription reaction primer, the thermostable DNA polymerase, PCR primer, dNTP mix (deoxyribonucleotide 5′-triphosphate; mixture of dATP, dGTP, dCTP and dTTP) and buffer. An RNA degrading enzyme inhibitor may be added to the reaction solution. As the reverse transcription reaction primer, a primer specific to the sequence of the target RNA, an oligo (dT) primer or a random primer can be used. As PCR primers, a primer pair (forward and reverse) specific to the sequence of cDNA generated by the reverse transcription reaction is used. The PCR primer may be the same as the reverse transcription reaction primer specific to the sequence of the target RNA. In addition, two or more kinds of PCR primers may be added to the 1-step RT-PCR reaction solution depending on the number of DNA regions to be amplified, that is, target sequences. If the test target is a norovirus, as a composition containing the above components, a mixture obtained by mixing NoV Reagents A, B and C contained in a commercially available norovirus detection reagent kit (Probe method) (Shimadzu Corp., product No. 241-09325 series, 241-09325-91 or 241-09325-92) according to the kit instruction manual can be used as 1-step RT-PCR reaction solution. As described above, elements added to purified water, saline or buffer which are internal control DNA or a forward or reverse primer that specifically hybridizes to the DNA, and which is used in the step (1) are removed.

When detecting norovirus RNA, for example, by using the PCR primers described in Patent Documents 1 and 2, Non-Patent Document 3, and Japanese Patent Publication 2018-78806, genogroup I (GI) and genogroup II (GII) in norovirus genotypes can be detected, but are not limited thereto. The norovirus detection reagent kit (probe method) contains the PCR primers described in Non-Patent Document 3.

In one embodiment of the present invention, when the specimen processing liquid in the step (3) contains SDS as a surfactant, if SDS having a strong denaturing effect on proteins is brought into the step (4) at a high concentration, there is a possibility that the enzymatic activities of the reverse transcriptase and the DNA polymerase contained in the 1-step RT-PCR reaction solution are inhibited, and RT-PCR does not proceed. Similarly, in one embodiment of the present invention, when the specimen processing liquid in the step (3) contains a hydroxide, if the hydroxide concentration introduced in the step (4) is high, the enzyme activity due to high pH is decreased. Therefore, in the step (4), the mixing ratio of the mixed solution obtained in the step (3) and the 1-step RT-PCR reaction solution is preferably 1:2 to 6, and more preferably 1:4, in volume ratio.

A person skilled in the art can easily set the reaction temperature conditions for the reverse transcription reaction in RT-PCR and the PCR conditions (temperature, time and number of cycles).

In one embodiment of the present invention, the RT-PCR product generated by the RT-PCR reaction in the step (5) is monitored by real-time determination. When the real-time determination is performed, RT-PCR and the step of detecting the RT-PCR product are performed in the same container.

Real-time determination of PCR products is also called real-time PCR. In real-time PCR, PCR amplification products are usually detected by fluorescence. The fluorescence detection method includes a method using an intercalating fluorescent dye and a method using a fluorescence-labeled probe. As the intercalating fluorescent dye, SYBR® Green I is used, but it is not limited thereto. The intercalating fluorescent dye binds to the double-stranded DNA synthesized by PCR and emits fluorescence upon irradiation with excitation light. By determining the fluorescence intensity, the amount of PCR amplification product produced can be measured. Besides, a temperature dissociation curve analysis may be performed to measure the peak detection temperature (Tm value of nucleic acid).

Fluorescently labeled probes include, but are not limited to, TaqMan probes, Molecular Beacon, cycling probes and the like. The TaqMan probe is an oligonucleotide modified at the 5′end with a fluorescent dye and at the 3′end with a quencher substance. The TaqMan probe hybridizes specifically to the template DNA in the annealing step of PCR, since the quencher exists on the probe, the generation of fluorescence is suppressed even when the excitation light is irradiated. In the subsequent extension reaction step, when the TaqMan probe hybridized to the template DNA is decomposed by the 5′→3′ exonuclease activity of Taq DNA polymerase, the fluorescent dye is released from the probe, the suppression of the fluorescence generation by the quencher is released, and fluorescence is emitted. By determining the fluorescence intensity, the amount of amplification product produced can be measured. Examples of the fluorescent dye include, but are not limited to, FAM, ROX, and Cy5. Examples of the quenchers include, but are not limited to, TAMRA® and MGB. In order to detect two or more types of DNA target sequences separately, PCR is performed using two or more types of oligonucleotide probes (for example, TaqMan probe) to which different fluorescent dyes are bound.

In the real-time determination of the PCR product, the amplification curve of the PCR product is monitored using a fluorescent filter corresponding to the fluorescent dye used. When the fluorescence intensity increases according to the number of PCR cycles, the presence of the gene to be analyzed in the specimen is determined to be positive. On the other hand, when the fluorescence intensity does not increase in PCR, it is determined to be negative. In the temperature dissociation curve analysis, when a predetermined temperature peak is observed, the presence of the gene to be analyzed is determined to be positive; when the predetermined temperature peak is not observed, the presence of the gene to be analyzed is determined to be negative.

In one embodiment of the present invention, a kit for detecting RNA virus by RT-PCR method is provided. The purified water, saline or buffer contained in the kit and used to suspend the specimen contain at least one element selected from the group consisting of internal control DNA, forward and reverse primers that specifically hybridize to the DNA. On the other hand, the 1-step RT-PCR reaction solution contained in the kit does not contain at least one element selected from the group consisting of internal control DNA, forward and reverse primers that specifically hybridize to the DNA, which are contained in the purified water, saline, or buffer. Therefore, unless the specimen processing liquid containing the specimen and the 1-step RT-PCR reaction solution is mixed, amplification of the internal control DNA is not observed, and at the same time, amplification of nucleic acid derived from the specimen is not observed. The test in which such a result is obtained is determined to be a test in which the RT-PCR reaction did not proceed or a test in which the specimen was not subjected to the RT-PCR reaction due to an artificial error. Accordingly, it is indicated that retesting of the specimen is necessary.

### EXAMPLES

Next, the present invention will be described in detail with reference to examples. However, the scope of the present invention is not limited thereby.

### Example 1

### (1) Specimens

100 mg of feces of a norovirus-infected patient as a specimen was collected and suspended in 1 mL of distilled water to prepare a fecal emulsion of about 10% (w/v). In the distilled water used, internal control DNA and forward and reverse primers that specifically hybridize to the DNA included in NoV Reagent B of Norovirus Detection Reagent Kit (Probe method) (Shimadzu Corp., product No. 241-09325 series) were previously added. The obtained fecal emulsion was centrifuged at 10,000 rpm for 5 minutes with a microcentrifuge to obtain a centrifugal supernatant.

### (2) Specimen Treatment

The following components were added to distilled water to prepare a specimen processing liquid.

50 mM sodium hydroxide (NaOH),

0.1% (w/v) sodium dodecyl sulfate (SDS), and

625 μM dNTP (dATP, dGTP, dCTP and dTTP)

The specimen treatment was carried out by taking 4 μL of the specimen processing liquid in a PCR reaction tube without a lid, adding 1 μL of the supernatant of the fecal emulsion obtained in (1) to the mixture, and then leaving it at room temperature for 3 minutes.

### (3) 1-Step RT-PCR Reaction

To a PCR reaction tube containing 5 μL of the mixed solution of the supernatant of the fecal emulsion and the specimen processing liquid obtained in (2), 20 μL of RT-PCR reaction solution which does not contain internal control DNA, forward and reverse primers that specifically hybridize to the DNA, and which is 1-step RT-PCR reaction solution prepared to have the following reaction solution composition, was added, mixed by stirring, and then spun down with a small centrifuge. Then, the RT-PCR reaction was immediately monitored using a real-time PCR device (GVP-9600, Shimadzu Corp.). Regarding the reaction, after reverse transcription reaction at 45° C. for 5 minutes, initial denaturation at 95° C. for 3 minutes was performed, and then PCR at 95° C. for 1 second-56° C. for 10 seconds was performed for 45 cycles to measure an amplification curve. Photometry in PCR was performed at a step of 56° C. for 10 seconds.

(Composition of Reaction Solution)
40 mM Tris
0.025 unit/μL Taq polymerase
1 unit/μL reverse transcriptase
3.75 mM magnesium chloride
400 nM PCR primer set (COG1F/COG1R and COG2F/COG2R) (see Non-Patent Document 3, Table 11)
200 nM fluorescent labeled probe (TaqMan probe: G1A, G1B and G2)

### (4) Results

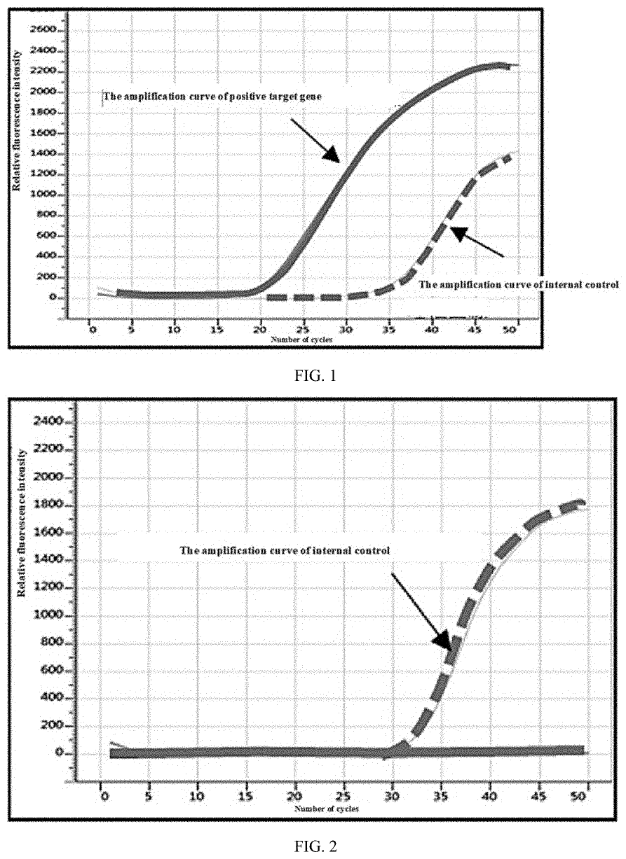

The measurement results are shown in FIG. 1. Since the amplification curve assigned to the internal control DNA was detected, it was shown that the PCR reaction was in progress. Further, since the amplification curve attributed to the norovirus gene is detected, the specimen subjected for the test is judged to be positive for norovirus.

### Example 2

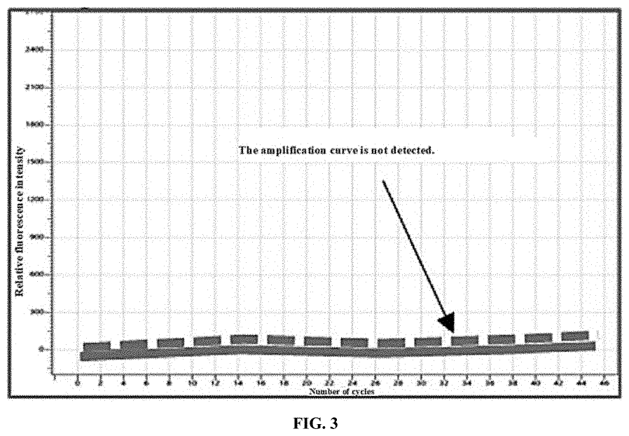

The test was performed in the same manner as in Example 1 except that the feces of a healthy person who was not infected with the norovirus was used. The measurement results are shown in FIG. 2. From FIG. 2, it was shown that the PCR reaction was in progress because the amplification curve assigned to the internal control DNA was detected. On the other hand, since the amplification curve attributed to the norovirus gene was not detected, the specimen used for the test is judged to be negative for norovirus.

### Example 3

The case where the centrifugal supernatant of the fecal emulsion prepared in Example 1 was not subjected to the RT-PCR reaction was examined. A 1-step RT-PCR reaction solution which does not contain internal control DNA, forward and reverse primers specifically hybridizing to the DNA was prepared in the same manner as in Example 1. The 1-step RT-PCR reaction solution alone was added to the PCR reaction tube containing only the specimen processing liquid described in Example 1 and the reaction was carried out in the same manner as in Example 1.

The measurement results are shown in FIG. 3. From the result that the amplification curve attributed to the norovirus gene was not detected, it is judged to be negative for norovirus. However, no amplification curve assigned to the internal control DNA is detected, indicating that the centrifugation supernatant of the fecal emulsion has not been added to the RT-PCR reaction. That is, it is indicated that the specimen has not been subjected to the test. Therefore, the above-mentioned judgement of negative norovirus is a false negative, which means that the specimen needs to be retested.

1. A method for detecting RNA virus in a specimen, comprising
(1) a step of suspending the specimen in purified water, saline or buffer which contain at least one element selected from the group consisting of internal control DNA, forward and reverse primers that specifically hybridize to the DNA;
(2) a step of extracting the centrifugal supernatant of the suspension obtained in the step (1);
(3) a step of mixing the centrifuge supernatant extracted in the step (2) with specimen processing liquid;
(4) a step of mixing the mixed solution obtained in the step (3) with 1-step RT-PCR reaction solution, which contains reverse transcriptase and DNA polymerase, but does not contain the element(s) selected in the step (1), and performing RT-FOR; and
(5) a step of detecting the RT-PCR product.

2. The method according to claim 1, wherein the specimen is derived from samples selected from the group consisting of biological samples, biologically derived samples, environmental samples, and environmentally derived samples.

3. The method according to claim 1, wherein the specimen is derived from samples selected from the group consisting of excretion samples, samples derived from excretion, vomitus and samples derived from vomitus.

4. The method according to claim 1, wherein the RNA virus is norovirus.

5. The method according to claim 4, wherein the genotype of the norovirus is genogroup I (GI) or genogroup II (GII).

6. The method according to claim 1, wherein the specimen processing liquid in the step (3) contains at least one type of surfactant.

7. The method according to claim 6, wherein the surfactant is an anionic surfactant.

8. The method according to claim 7, wherein the anionic surfactant is selected from the group consisting of alkyl sulphates, alkyl ether sulphates, docusates, sulphonate fluorosurfactants, alkylbenzene sulphonates, alkylaryl ether phosphates, alkyl ether phosphates, alkylcarboxylates, sodium lauroyl sarcosine, carboxylate fluorosurfactants, sodium cholate and sodium deoxycholate.

9. The method according to claim 7, wherein the anionic surfactant is an alkyl sulphate.

10. The method according to claim 9, wherein the alkyl sulfate is sodium dodecyl sulfate or ammonium dodecyl sulfate.

11. The method according to claim 6, wherein the concentration of the anionic surfactant is 0.02 to 0.5% (w/v).

12. The method according to claim 1, wherein the specimen processing liquid in the step (3) contains a hydroxide.

13. The method according to claim 12, wherein the hydroxide is sodium hydroxide or potassium hydroxide.

14. The method according to claim 12, wherein the concentration of the hydroxide is 10 to 100 mM.

15. The method according to claim 1, wherein the mixing ratio of the centrifugal supernatant and the specimen processing liquid in the step (3) is 1:3 to 6 as a volume ratio.

16. The method according to claim 1, wherein the specimen processing liquid in the step (3) is the sample treatment reagent contained in the norovirus detection reagent kit (probe method) (Shimadzu Corp., product No. 241-09325 series).

17. The method according to claim 1, wherein the 1-step RT-PCR reaction solution in the step (4) is a mixture of NoV Reagents A, B and C contained in the norovirus detection reagent kit (probe method) (Shimadzu Corp., product No. 241-09325 series), which does not contain the element(s) selected in the step (1).

18. The method according to claim 1, wherein the reverse transcriptase is selected from the group consisting of AMV reverse transcriptase, MMLV reverse transcriptase, HIV reverse transcriptase and variants thereof.

19. The method according to claim 1, wherein the DNA polymerase is selected from the group consisting of Taq DNA polymerase, Tth DNA polymerase, KOD DNA polymerase, Pfu DNA polymerase and variants thereof.

20. The method according to claim 1, wherein detection of the RT-PCR product in the step (5) is monitored by real-time determination.

21. The method according to claim 20, wherein in the real-time determination, the presence of RNA in a specimen is judged to be positive or negative by measuring the peak in the amplification curve or melting curve of the RT-PCR product using a fluorescent filter.

22. The method according to claim 20, wherein in the real-time determination, whether a specimen needs to be retested is judged by measuring the peak in the amplification curve or melting curve of the PCR product against the internal control DNA.

23. A kit for detecting RNA virus in a specimen, comprising
(1) purified water, saline or buffer which contain at least one element selected from the group consisting of internal control DNA, forward and reverse primers that specifically hybridize to the DNA;
(2) specimen processing liquid; and
(3) 1-step RT-PCR reaction solution which contains reverse transcriptase and DNA polymerase, but does not contain the element(s) selected in the step (1).

24. The kit according to claim 23, wherein the reverse transcriptase is selected from the group consisting of AMV reverse transcriptase, MMLV reverse transcriptase, HIV reverse transcriptase and variants thereof.

25. The kit according to claim 23, wherein the DNA polymerase is selected from the group consisting of Taq DNA polymerase, Tth DNA polymerase, KOD DNA polymerase, Pfu DNA polymerase and variants thereof.

26. The kit according to claim 23, wherein the specimen processing liquid contains at least one type of surfactant.

27. The kit according to claim 26, wherein the surfactant is an anionic surfactant.

28. The kit according to claim 27, wherein the anionic surfactant is selected from the group consisting of alkyl sulphates, alkyl ether sulphates, docusates, sulphonate fluorosurfactants, alkylbenzene sulphonates, alkylaryl ether phosphates, alkyl ether phosphates, alkylcarboxylates, sodium lauroyl sarcosine, carboxylate fluorosurfactants, sodium cholate and sodium deoxycholate.

29. The kit according to claim 27, wherein the anionic surfactant is an alkyl sulphate.

30. The kit according to claim 29, wherein the alkyl sulfate is sodium dodecyl sulfate or ammonium dodecyl sulfate.

31. The kit according to claim 23, wherein the specimen processing liquid contains a hydroxide.

32. The kit according to claim 31, wherein the hydroxide is sodium hydroxide or potassium hydroxide.

33. The kit according to claim 23, wherein the specimen processing liquid is a sample treatment reagent contained in the norovirus detection reagent kit (probe method) (Shimadzu Corp., product No. 241-09325 series).

34. The kit according to claim 23, wherein the 1-step RT-PCR reaction solution is a mixture of NoV Reagents A, B and C contained in the norovirus detection reagent kit (probe method) (Shimadzu Corp., product No. 241-09325 series), but does not contain the element(s) selected in (1).
